SEQUENCE LISTING (6 sequences)

SEQ ID NO: 1 - protein, 10 aa, synthetic construct
GRRRRELDPM

SEQ ID NO: 2 - protein, 9 aa, synthetic construct
RQRRELDPM

SEQ ID NO: 3 - protein, 8 aa, synthetic construct
KIEAVKSE

SEQ ID NO: 4 - protein, 8 aa, synthetic construct
KIEAVQSE

SEQ ID NO: 5 - protein, 8 aa, synthetic construct
VGCGVLLS

SEQ ID NO: 6 - protein, 7 aa, synthetic construct
GCGVLLS

(19) United States
(12) Patent Application Publication
(10) Pub. No.: US 2024/0344025 A1
(43) Pub. Date: Oct. 17, 2024

(54) SYNTHETIC IMMUNO-SUPPRESSIVE CELLS AND METHODS OF USE THEREOF

(71) Applicant: The Regents of the University of California, Oakland, CA (US)

(72) Inventors: Wendell A. LIM, San Francisco, CA (US); Nishith REDDY, San Francisco, CA (US); Milos SIMIC, San Francisco, CA (US)

(21) Appl. No.: 18/294,854

(22) PCT Filed: Sep. 21, 2022
(86) PCT No.: PCT/US2022/076797

§ 371 Date: Feb. 2, 2024

Related U.S. Application Data
(60) Provisional application No. 63/246,728, filed on Sep. 21, 2021.

(51) Int. Cl. C12N 5/0783; A61K 35/17; A61P 37/06; C07K 14/54; C07K 14/55; C07K 14/705; C07K 16/24

(52) U.S. Cl. CPC C12N 5/0636; A61K 35/17; A61P 37/06; C07K 14/5428; C07K 14/55; C07K 14/70596; C07K 16/24; C12N 2510/00

(57) ABSTRACT

Provided herein is a synthetic immuno-suppressive cell that contains comprises a molecular circuit comprising the following components: (a) a binding-triggered transcriptional switch (BTTS); and one or both of: (b) a nucleic acid encoding a pro-inflammatory cytokine sink, (c) a nucleic acid encoding an anti-inflammatory cytokine, (d) a nucleic acid encoding an immune inhibitory receptor, or ligand thereof, and (c) a nucleic acid encoding an ectonucleotidase. In this circuit, binding of the BTTS to a marker on the surface of a target cell activates expression of one or any combination of (b)-(c) by the synthetic immuno-suppressive cell.

### CROSS-REFERENCING

This application claims the benefit of provisional application Ser. No. 63/246,728, filed on Sep. 21, 2021, which application is incorporated by reference herein in its entirety.

### STATEMENT REGARDING FEDERALLY SPONSORED RESEARCH

This invention was made with government support under grant no. DK116264 awarded by The National Institutes of Health. The government has certain rights in the invention.

### INCORPORATION BY REFERENCE OF SEQUENCE LISTING PROVIDED AS A TEXT FILE

A Sequence Listing is provided herewith as a Sequence Listing XML, “UCSF-660WO_SEQ_LIST” created on Sep. 18, 2022 and having a size of 7 KB. The contents of the Sequence Listing XML are incorporated by reference herein in their entirety.

### INTRODUCTION

Many autoimmune or inflammatory diseases involve the hyperactivation of immune cells in particular tissues. In the body, T regulatory cells (Tregs) often keep autoreactive immunity in check by acting as suppressor cells. Tregs are thought to act by producing suppressive cytokines and by consuming proliferative cytokines. They may act in disease tissues or the draining lymph node. Tregs can be used as a cellular therapy for autoimmune disease. However, several major challenges remain—how to direct T regs to a disease site, how to maintain a stable T reg fate (vs conversion to inflammatory T cells), and how to engineer/manufacture Tregs. There is a great need for solutions to these problems.

### SUMMARY

The present disclosure describes a way to locally dampen inflammation at targeted sites. The strategy can be implemented using conventional CD4⁺ T cells that have been engineered to provide immunosuppressive signals. However, other immune cells, including macrophages, can be used. These cells can be engineered to sense antigens at the affected site using a binding triggered transcriptional switch (a BTTS) and in response to induce expression/production of suppressive cytokines, inflammatory cytokine sinks, suppressor molecules, or any combinations thereof.

In some embodiments, the engineered immune cell may comprise a molecular circuit comprising the following components: (a) a binding-triggered transcriptional switch and one or both of: (b) a nucleic acid encoding a pro-inflammatory cytokine sink (e.g., a protein comprising the extracellular domain of IL-1R, IL-2R/CD25, IL-12R, IL-18R, TNFR1, TNFR2, IFNGR, GM-CSFR), (c) a nucleic acid encoding an anti-inflammatory cytokine (e.g., Il-1ra, IL-4, IL-6, IL-10, IL-11, IL-13 and TGF-β, or a variant thereof), (d) a nucleic acid encoding an immune inhibitory receptor, or ligand thereof; and (e) a nucleic acid encoding an ectonucleotidase.

In this circuit, binding of the binding-triggered transcriptional switch to a marker on the surface of a target cell activates expression of one or any combination of (b)-(e) by the engineered immune cell. For example, binding of the binding-triggered transcriptional switch to a marker on the surface of a target cell may activate expression of the pro-inflammatory cytokine sink of (b) and/or the anti-inflammatory cytokine of (c) by the engineered immune cell.

Examples of such circuits and their use are described in further detail below.

### BRIEF DESCRIPTION OF THE DRAWINGS

FIG. 1. shows synNotch induced production of suppressive cytokine TGFb.

FIGS. 2A and 2B show that suppressor cells that produce combination of TGFb (suppressive cytokine) and CD25 (IL2 sink) are very effective at suppressing CAR T killing in vitro.

FIGS. 3A and 3B show that suppressor cells that produce combination of IL10 (suppressive cytokine) and CD25 (IL2 sink) are very effective at suppressing CAR T killing in vitro.

FIGS. 4A and 4B shows that suppressor cells that produce combination of TGFb (suppressive cytokine) and CD25 (IL2 sink) are very effective at suppressing CAR T killing of tumors in vivo.

FIG. 5. shows that engineered T cells overexpressing CD25 increases consumption of IL2 and cell proliferation.

FIG. 6. shows that synNotch→IL10 synthetic suppressor cells can block autoimmune cell proliferation in brain and CNS in mouse neuroinflammation model.

### DEFINITIONS

As used herein, the terms “treatment,” “treating,” “treat” and the like, refer to obtaining a desired pharmacologic and/or physiologic effect and/or a response related to the treatment. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent (including biologic agents, such as cells), or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

As used herein, the term “binding-triggered transcriptional switch” or “BTTS” refers to any polypeptide or complex of the same that is capably of transducing a specific binding event on the outside of the cell (e.g., binding of an extracellular domain of the BTTS) to activation of a recombinant promoter within the nucleus of the cell. Many BTTSs work by releasing a transcription factor that activates the promoter. In these embodiments, the BTTS is made up of one or more polypeptides that undergo proteolytic cleavage upon binding to the antigen to release a gene expression regulator that activates the recombinant promoter. For example, a BTTS may comprise (i) an extracellular domain comprising the antigen binding region of an antigen-specific antibody; (ii) a proteolytically cleavable sequence comprising one or more proteolytic cleavage sites; and (iii) an intracellular domain, wherein binding of the antigen binding region to the antigen induces cleavage of the sequence at the one or more proteolytic cleavage sites, thereby releasing the intracellular domain and wherein the intracellular domain activates transcription of an expression cassette. A BTTS can be based on synNotch, A2, MESA, or force receptor, for example, although others are known or could be constructed.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113. Rosenburg and Moore eds., Springer-Verlag, New York, pp. 269-315 (1994).

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA. DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “vector” or “expression vector” is a replicon, such as plasmid, phage, virus, or cosmid, to which another DNA segment, i.e. an “insert”, may be attached so as to bring about the replication of the attached segment in a cell.

The term “heterologous”, as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an.” and “the” include plural referents unless the context clearly dictates otherwise. Thus, for example, reference to “a cell” includes a plurality of such cells and reference to “the cell” includes reference to one or more cells and equivalents thereof known to those skilled in the art, and so forth. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, the present disclosure provides a synthetic immune suppressor cell that locally dampens inflammation at targeted sites. In some embodiments, the engineered immune cell may comprise a molecular circuit comprising the following components: (a) a binding-triggered transcriptional switch (BTTS) and one any combination of: (b) a nucleic acid encoding a pro-inflammatory cytokine sink (e.g., a protein comprising the extracellular domain of IL-1R, IL-2R/CD25, IL-12R, IL-18R, TNFR1, TNFR2, IFNGR, GM-CSFR), (c) a nucleic acid encoding an anti-inflammatory cytokine (e.g., Il-1ra, IL-4, IL-6, IL-10, IL-11, IL-13, IL-35 and TGF-β, or a variant thereof), (d) a nucleic acid encoding an immune inhibitory receptor, e.g., PD1, CTLA4, BTLA, CD160, KRLG-1, 2B4, Lag-3, Tim-3, or TIGIT, or ligand thereof such as PDL1; and (e) a nucleic acid encoding an ectonucleotidase (e.g., CD39 or CD73).

In this circuit, binding of the BTTS to a marker on the surface of a target cell activates expression of one or any combination of (b)-(e) by the engineered immune cell. Specifically, binding of the BTTS to a marker on the surface of a target cell may activate expression of (a), (b), (c), (d), (c). (a) and (b), (a) and (c), (a) and (d), (a) and (e), (b) and (c), (b) and (d), (b) and (c), (c) and (d), (c) and (e) or (d) and (e), etc., by the engineered immune cell For example, binding of the BTTS to a marker on the surface of a target cell activates expression of the anti-inflammatory cytokine of (b) and/or the pro-inflammatory cytokine sink of (c) by the engineered immune cell.

### Binding-Triggered Transcriptional Switches (BTTSs)

The BTTS is a cleavable fusion protein contains: (a) an extracellular binding domain comprising a protein binding domain (e.g., scFv or nanobody) that binds to cell surface marker on a cell, (b) a force sensing region. (c) a transmembrane domain, (d) one or more force-dependent cleavage sites that are cleaved when the force sensing region is activated, and (e) an intracellular domain comprising a transcriptional activator, where binding of the binding domain to the marker on the surface of the other cell induces proteolytic cleavage of the one or more force-dependent cleavage sites to release the transcriptional activator.

In this switch, the fusion protein is cleaved to release the intracellular domain when the extracellular domain of the fusion protein engages with a marker on another cell. As such, in many cases, the fusion protein will contain a force sensing region (which is typically in the extracellular domain) and one or more force-dependent cleavage sites that are cleaved when the force sensing region is activated. The position of the force-dependent cleavage sites may vary and, in some embodiments the fusion protein may contain at least two cleavage sites. In some cases, one of the cleavage sites may be extracellular and the other may be in the transmembrane domain or within 10 amino acids of the transmembrane domain in the intracellular domain. In any embodiment, the force sensing region and/or the one or more force-dependent cleavage sites may be from a Delta/Serrate/Lag2 (DSL) superfamily protein, as reviewed by Pintar et al (Biology Direct 2007 2:1-13). For example, the force sensing region and/or the one or more force-dependent cleavage sites may be from Notch (see Morsut Cell. 2016 164:780-91), von Willebrand Factor (vWF), amyloid-beta, CD16, CD44, Delta, a cadherin, an ephrin-type receptor or ephrin ligand, a protocadherin, a filamin, a synthetic E cadherin, interleukin-1 receptor type 2 (ILIR2), major prion protein (PrP), a neuregulin or an adhesion-GPCR. Several other examples of this type of protein are known and listed in Pintar, supra. Many members of this family appear to share a similar architecture a region that unfolds and opens up a protease cleavage site (e.g., EGF-like repeats; see Cordle et al Nat. Struct. Mol. Biol. 2008 15:849-857), a trans-membrane segment, and a relatively short (˜100-150 amino acids) intracellular domain.

These sequences permit the binding-triggered release of a transcriptional activator from the membrane in their natural environment and can be readily adapted herein.

In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence. The S3 proteolytic cleavage site is in the transmembrane domain. In many cases, the shear force generated by binding of the extracellular domain of this fusion protein to another cells unfolds the force sensing region (which, in the case of Notch contains EGF-like repeats whereas in other protein is made up of other sequences such as the A2 domain in vWF (see, e.g., J Thromb Haemost. 2009 7:2096-105, Lippok Biophys J. 2016 110:545-54, Lynch Blood. 2014 123:2585-92, Crawley, Blood. 2011 118:3212-21 and Xy J Biol Chem. 2013 288:6317-24) or modified A2 domain that has, e.g., the R1597W, E1638K and I1628T substitutions. The architecture of such proteins is described in, e.g., Morsut et al, Cell. 2016 164:780-91. WO2016138034 and WO2019099689, among other places).

In some cases, the fusion protein includes an S1 ligand-inducible proteolytic cleavage site. An S1 ligand-inducible proteolytic cleavage site can be located between the HD-N segment and the HD-C segment. In some cases, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some cases, an amino acid sequence comprising an S1 ligand-inducible proteolytic cleavage site can have the amino acid sequence GRRRRELDPM (SEQ ID NO:1), where cleavage occurs between the “RE” sequence. As another example, an amino acid sequence comprising an S1 ligand-inducible proteolytic cleavage site can have the amino acid sequence RQRRELDPM (SEQ ID NO:2), where cleavage occurs between the “RE” sequence.

In some cases, the fusion protein polypeptide includes an S2 ligand-inducible proteolytic cleavage site. An S2 ligand-inducible proteolytic cleavage site can be located within the HD-C segment. In some cases, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 ligand-inducible proteolytic cleavage site can have the amino acid sequence KIEAVKSE (SEQ ID NO:3), where cleavage occurs between the “AV” sequence. As another example, an amino acid sequence comprising an S2 ligand-inducible proteolytic cleavage site can have the amino acid sequence KIEAVQSE (SEQ ID NO: 4), where cleavage occurs between the “AV” sequence.

In some cases, the fusion protein includes an S3 ligand-inducible proteolytic cleavage site. An S3 ligand-inducible proteolytic cleavage site can be located within the TM domain. In some cases, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO:5), where cleavage occurs between the “GV” sequence. In some cases, an S3 ligand-inducible proteolytic cleavage site comprises the amino acid sequence GCGVLLS (SEQ ID NO:6).

In some cases, the fusion protein polypeptide lacks an S1 ligand-inducible proteolytic cleavage site. In some cases, the BTTS lacks an S2 ligand-inducible proteolytic cleavage site. In some cases, the BTTS lacks an S3 ligand-inducible proteolytic cleavage site. In some cases, the BTTS lacks both an S1 ligand-inducible proteolytic cleavage site and an S2 ligand-inducible proteolytic cleavage site. In some cases, the BTTS includes an S3 ligand-inducible proteolytic cleavage site; and lacks both an S1 ligand-inducible proteolytic cleavage site and an S2 ligand-inducible proteolytic cleavage site.

In some embodiments, the fusion protein may have an vWF A2 sequence or a variation thereof, an ADAMTS13 cleavage site (which may be described by the consensus sequence HEXXHXXGXXHD; Crawley, Blood. 2011 118:3212-21), and an S3 or γ-secretase cleavage site, although many other arrangements exist. In some embodiments, the switch may contain components that are borrowed from Notch. In other embodiments, the switch may not contain components that are from Notch.

For simplicity, BTTSs, including but not limited to chimeric notch receptor polypeptides, are primarily single polypeptide chains. However, BTTSs, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTSs that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851: the disclosure of which is incorporated herein by reference in its entirety.

Useful BTTSs that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105 (1): 64-9); the disclosure of which is incorporated herein by reference in its entirety.

Useful BTTSs that may be employed in the subject methods include, but are not limited to von Willebrand Factor (vWF) cleavage domain-based BTTSs, such as but not limited to e.g., those containing a unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTSs are described in Langridge & Struhl (Cell (2017) 171 (6): 1383-1396); the disclosure of which is incorporated herein by reference in its entirety.

Useful BTTSs that may be employed in the subject methods include, but are not limited to chimeric Notch receptor polypeptides, such as but not limited to e.g., synNotch polypeptides, non-limiting examples of which are described in PCT Pub. No. WO 2016/138034, U.S. Pat. Nos. 9,670,281, 9,834,608, Roybal et al. Cell (2016) 167 (2): 419-432. Roybal et al. Cell (2016) 164 (4): 770-9, and Morsut et al. Cell (2016) 164 (4): 780-91; the disclosures of which are incorporated herein by reference in their entirety. The “SNIPR” switch is another example of a BTTS (see Zhu et al, bioRxiv 2021), although others exist and/or can be readily designed.

Expression of the anti-FAP BTTS in the cell may be constitutive or inducible, e.g., by binding of another BTTS to an antigen on another cell in the patient.

Examples of transcriptional activators that can be part of the fusion protein are numerous and include artificial transcription factors (ATFs) such as, e.g., Zinc-finger-based artificial transcription factors (including e.g., those described in Sera T. Adv Drug Deliv Rev. 2009 61 (7-8): 513-26; Collins et al. Curr Opin Biotechnol. 2003 14 (4): 371-8; Onori et al. BMC Mol Biol. 2013 14:3. In some cases, the transcriptional activator may contain a GAL4 DNA binding domain, which binds to the Gal4 responsive UAS, which has been well characterized in the art. Examples of suitable transcriptional activators include GAL4-VP16 and GAL4-VP64, although many others could be used. As would be appreciated, the identity of the transcription activators may vary. In some embodiments, the transcription factor may have a DNA binding domain that binds to a corresponding promoter sequence and an activation domain. In many embodiments, the DNA binding domain transcription factor may be independently selected from Gal4-, LexA-, Tet-, Lac-, dCas9-, zinc-finger- and TALE-based transcription factors. TALE- and CRISPR/dCas9-based transcription factors are described in Lebar (Methods Mol Biol. 2018 1772:191-203), among others. The binding sites for such domains are well known or can be designed at will. The transcription factors can have any suitable activation domain, e.g., VP16, VP64, Ela, Sp1, VP16, CTF, GAL4 among many others.

### Extracellular Binding Domains

The extracellular binding domain of the BTTS may bind to a tissue- or organ-specific cell-surface marker, a disease-specific cell-surface marker, or an off-target cell-surface marker, depending on how the cell is being used. For example, if one wanted to dampen immune cell mediated responses in the brain and/or spinal cord (e.g., as a result of a disease such as multiple sclerosis), then the BTTS may have an extracellular domain that binds to a brain and/or CNS-specific cell-surface marker (e.g., MOG, CDH10, BCAN, CSPG5, PTPRZ1 or NRCAM) which are both preferentially expressed in the brain). Likewise if one wanted to dampen immune cell mediated responses in the pancreas, then the BTTS may have an extracellular domain that binds to a pancreatic cell surface marker (e.g., GP2, CD133, ion transport regulator 2 (FXYD2), tetraspanin 7 (TSPAN7), transmembrane protein 27 (TMEM27), discoidin domain receptor tyrosine kinase 1 (DDR1) and delta/notch-like EGF repeat containing (DNER), dispatched homologue 2 (DISP2), seizure related 6 homologue like (SEZ6L2), low density lipoprotein receptor-related protein 11 (LRP11), HEPACAM family member 2 (HEPACAM2), TSPAN7 and TMEM27, etc.) Tissue-specific cell-surface markers are available for the eye, retina, heart, skeletal muscle, smooth muscle, adrenal gland, parathyroid gland, thyroid gland, pituitary gland, lung, bone marrow, lymphoid tissue, liver, gallbladder, testis, epididymis, prostate, seminal vesicle, ductus deferens, adipose tissue, brain, salivary gland, esophagus, tongue, stomach, intestine, pancreas, kidney, urinary bladder, breast, vagina, cervix, endometrium, fallopian tube, ovary, placenta, skin, blood, etc. In another example, the extracellular binding domain may bind to a tissue or organ-specific cell surface marker in a transplanted organ (e.g., pancreas, liver, lung, or kidneys, etc.), thereby protecting it from attack from killer T cells. In any embodiment, the BTTS may bind to CD19, for example.

Likewise, if one wanted to dampen “off-target” immune responses (which may occur when cell therapies, e.g., CAR T therapies, attack sites that are off target) the extracellular binding domain may bind to a marker in the off-target sites. As would be apparent, this marker may vary depending on the therapy being used. However, many off-target markers are list as “NOT” antigens in Dannenfelser (Cell Syst. 2020 11:215-228) WO 2017/193059, WO 2020/097395 and PCT/US2021/045796).

Finally, if one wanted to dampen immune cell mediated responses in an inflamed tissue, then the BTTS may have an extracellular domain that binds to an inflammation-specific marker. Non-limiting examples of antigens associated with inflammatory disease include, e.g., AOC3 (VAP-1), CAM-3001, CCL11 (cotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7. LFA-1 (CD11a), myostatin, OX-40, scleroscin, SOST, TGF beta 1. TNF-α, and VEGF-A. Antigens for other diseases may be targeted in the same way.

### Anti-Inflammatory Cytokines

If the protein induced by the BTTS binding to FAP is an anti-inflammatory cytokine, the cytokine will be secreted from the cell. In these embodiments, the circuit may comprise a nucleic acid containing a promoter that is activated by the released transcriptional activator, and a coding sequence encoding an anti-inflammatory cytokine. In this disclosure, the term “anti-inflammatory cytokine” is intended to encompass natural molecules that have anti-inflammatory activity (e.g., Il-1ra, IL-4, IL-6, IL-10, IL-11, IL-13. IL-35 and TGF-β), as well as non-natural or “engineered” cytokines that have anti-inflammatory activity. As would be appreciated, cytokines are secreted from the cell and their coding sequence will encode a secretion signal.

### Pro-Inflammatory Cytokine Sinks

The term “pro-inflammatory cytokine sink” is intended to refer to a protein that specifically binds to a pro-inflammatory cytokine (e.g., IL-2, CCL-21, IL-12, IL-7, IL-15 or IL-21, etc.) and prevents it from binding with its cognate receptor on another immune cell. In some embodiments, the cytokine sink comprises at least the extracellular domain of a receptor for a pro-inflammatory cytokine, e.g., at least the extracellular domain of IL-1R, IL-2R/CD25, IL-12R. IL-18R, TNFR1, TNFR2, IFNGR, GM-CSFR, etc., or a part thereof that binds to its cognate ligand. For example, the cytokine sink may have the extracellular domain of IL-1R (which binds to IL-1), IL-2R or CD25 (which binds to IL-2), IL-12R, IL-18R (which binds to IL-18), TNFR1 and TNFR2 (which binds to TNF-α), IFNGR (which binds to IFNγ) and GM-CSFR (which binds to GMCSF) or a subunit thereof that binds to its ligand. This domain may be tethered to the cell via a transmembrane domain or it may be secreted. If the domain is tethered to the cell then, in some embodiments, a truncated or mutated form of the receptor may be used so that the receptor is incapable of signaling. In other embodiments, the full-length receptor may be used. In these embodiments, the cell may not have the internal machinery to transduce a signal from that receptor to the nucleus. In one embodiment, sink may contain the extracellular domain of CD25 (which is the receptor for IL-2), although others could be used too.

In alternative embodiments, an antibody (e.g., a scFv) that binds to the pro-inflammatory cytokine may be used. In these embodiments, the antibody may be tethered to the cell, e.g., via a transmembrane domain, or secreted.

In these embodiments, the circuit may comprise a nucleic acid containing a promoter that is activated by the released transcriptional activator, and a coding sequence encoding a pro-inflammatory cytokine sink.

### Ectonucleotidase

Ectonucleotidases are nucleotide metabolizing enzymes that are expressed on the plasma membrane and have externally oriented active sites. These enzymes metabolize nucleotides to nucleosides. Extracellular adenosine generated by the ectonucleotidases CD39 and CD73 is a newly recognized “immune checkpoint mediator” that is believed to interfere with anti-tumor immune responses. Expressing an ectonucleotidase such as CD39 or CD73 on a cell should dampen the immune response around that cell. In these embodiments, the circuit may comprise a nucleic acid containing a promoter that is activated by the released transcriptional activator, and a coding sequence encoding a ectonucleotidase.

### Combinations

As noted above, in some embodiments expression of two or more of (a)-(e) may be induced by binding of the BTTS to the cell surface marker. In these embodiments, the different proteins may be on different constructs with the same promoter or their expression may be coordinated by an IRES. Other ways for co-expressing two proteins are known. The two or more of (a)-(e) may be on the same vector or different vectors.

### Cells

The cells employed herein are immune cells that contain one or more of the described nucleic acids, expression vectors, etc., encoding the desired components. Immune cells of the present disclosure include mammalian immune cells including, e.g., those that are genetically modified to produce the components of a circuit of the present disclosure or to which a nucleic acid, as described above, has been otherwise introduced. In some instances, the subject immune cells have been transduced with one or more nucleic acids and/or expression vectors to express one or more components of a circuit of the present disclosure.

Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual.

Such cells include, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells) and cytotoxic T-cells (CD8+ cells). A “cytotoxic cell” includes CD8+ T cells, natural-killer (NK) cells, and neutrophils, which cells are capable of mediating cytotoxicity responses. In any embodiment, the cell may be a CD4⁺ T cell (i.e., T helper cell) or a macrophage, for example.

Immune cells encoding a circuit of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, a primary is obtained from an individual; and the cell obtained from the individual is modified to express components of a circuit of the present disclosure. In other embodiments, an non-immunogenic allogeneic cell may be used.

### Circuits

As noted above, binding of BTTS the cell surface marker on another cell activates expression of one or more other protein. In these embodiments, binding of the binding domain of the BTTS to the antigen on the surface of a stromal cell induces proteolytic cleavage of the one or more force-dependent cleavage sites to release the transcriptional activator. The released transcriptional activator then binds to a promoter that drives the expression of the one or more other proteins, thereby inducing expression of the one or more other proteins. The general principles of a circuit are described in WO 2016/138034, U.S. Pat. Nos. 9,670,281, 9,834,608, Roybal et al. Cell (2016) 167 (2): 419-432, Roybal et al. Cell (2016) 164 (4): 770-9, and Morsut et al. Cell (2016) 164 (4): 780-91, among others.

### Methods of Treatment

A method of treatment is described below. In general terms, this method may comprise administering a cell described above to the subject. In some embodiments, primary immune cells may be purified from an individual, constructs encoding the above proteins may be introduced into the cells ex vivo, and the recombinant cells may be expanded and administered to the subject, e.g., by injection. In other embodiments, pre-made allogeneic cells (which may have abrogated MHC class I molecules) may be used instead.

In some embodiments, the subject may have cancer. In these embodiments, the subject may be receiving a course of cytotoxic immune cells (e.g., CAR T or NK cells) that kill cancer cells in an antigen-specific manner. In these embodiments, the present circuit may be in the cytotoxic immune cells or it may be in separate immune cells (e.g., CD8⁺ T cells or macrophage). In these embodiments, the BTTS can be targeted to off-target sites (i.e., normal tissue that is not cancerous), thereby providing a way to protect those sites. In these embodiments, the BTTS may an extracellular binding domain that binds to cells that are not part of the cancer. Exemplary cell surface markers for off-target sites may be listed as “NOT” antigens in Dannenfelser (Cell Syst. 2020 11:215-228) WO 2017/193059, WO 2020/097395 and PCT/US2021/045796), as described above.

In some embodiments, the subject may be a recipient of an organ transplant, e.g., a heart transplant, a lung transplant, a liver transplant, a pancreas transplant, a cornea transplant, a trachea transplant, a kidney transplant, a skin transplant, or a vascular tissue transplant. In these embodiments, the BTTS may have an extracellular binding domain that binds to cells, e.g., a tissue-specific antigen or organ-specific antigen, in the organ transplant.

In some embodiments, the subject may have a cytokine-induced condition induced by an infectious disease. In these embodiments, the BTTS may have an extracellular binding domain that binds to cells in the inflamed tissue. For example, in some embodiments, the patient may have ARDS, or organ-specific sepsis. In these embodiments, the circuit may be activated in the tissue/organ that is experiencing the condition, thereby reducing its effects.

In some embodiments, the subject may have a disease or condition that has local inflammation as part of its sequela, e.g., cardiovascular disease (which results in atherosclerotic plaque formation) and irritable bowel disease (IBD), although many others are known.

In some embodiments, the subject may have an autoimmune disease. In these embodiments, the BTTS may have an extracellular binding domain that binds to cells in the inflamed tissue, e.g., to a disease-specific antigen or to an organ/tissue specific antigen. Autoimmune diseases include, but are not limited to, achalasia, Addison's disease, adult still's disease, agammaglobulinemia, alopecia areata, amyloidosis, ankylosing spondylitis, anti-gbm/anti-tbm nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, autoimmune urticaria, axonal & neuronal neuropathy, Baló disease, Behcet's disease, benign mucosal pemphigoid, bullous pemphigoid, Castleman disease, celiac disease, Chagas disease, chronic inflammatory demyelinating polyneuropathy, chronic recurrent multifocal osteomyelitis, Churg-Strauss syndrome or eosinophilic granulomatosis, cicatricial pemphigoid, Cogan's syndrome, cold agglutinin disease, congenital heart block, coxsackie myocarditis, Crest syndrome, Crohn's disease, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, essential mixed cryoglobulinemia. Evans syndrome, fibromyalgia, fibrosing alveolitis, giant cell arteritis (temporal arteritis), giant cell myocarditis, glomerulonephritis, goodpasture's syndrome, granulomatosis with polyangiitis, Graves' disease. Guillain-Barre syndrome, Hashimoto's thyroiditis, hemolytic anemia, Henoch-Schonlein purpura, herpes gestationis or pemphigoid gestationis, hidradenitis suppurativa (acne inversa), hypogammalglobulinemia, iga nephropathy, IGg4-related sclerosing disease, immune thrombocytopenia purpura (itp), inclusion body myositis (ibm), interstitial cystitis (ic), juvenile arthritis, juvenile diabetes (type 1 diabetes), juvenile myositis (jm), kawasaki disease, lambert-eaton syndrome, leukocytoclastic vasculitis, lichen planus, lichen sclerosus, ligneous conjunctivitis, linear iga disease (lad), lupus, lyme disease chronic, meniere's disease, microscopic polyangiitis (mpa), mixed connective tissue disease (mctd), Mooren's ulcer, Mucha-Habermann disease, multifocal motor neuropathy, multiple sclerosis, myasthenia gravis, myelin oligodendrocyte glycoprotein antibody disorder, myositis, narcolepsy, neonatal lupus, neuromyelitis optica, neutropenia, ocular cicatricial pemphigoid, optic neuritis, palindromic rheumatism, pandas, paraneoplastic cerebellar degeneration (pcd), paroxysmal nocturnal hemoglobinuria. Parry Romberg syndrome, pars planitis (peripheral uveitis), Parsonage-Turner syndrome, pemphigus, peripheral neuropathy, perivenous encephalomyelitis, pernicious anemia, Poems syndrome, polyarteritis nodosa, polyglandular syndromes type i, ii, iii, polymyalgia rheumatica, polymyositis, postmyocardial infarction syndrome, postpericardiotomy syndrome, primary biliary cholangitis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, schmidt syndrome, scleritis, scleroderma, sjögren's syndrome, sperm & testicular autoimmunity, stiff person syndrome, subacute bacterial endocarditis, Susac's syndrome, sympathetic ophthalmia, Takayasu's arteritis, temporal arteritis/giant cell arteritis, thrombocytopenia purpura, thyroid eye disease, Tolosa-Hunt syndrome, transverse myelitis, type 1 diabetes, ulcerative colitis, undifferentiated connective tissue disease, uveitis, vasculitis, vitiligo, and Vogt-Koyanagi-Harada disease.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

In the following examples, synthetic suppressor cells have been shown to locally block CD8⁺ T cell-mediated killing of cancer cells highly effectively, in vitro and in vivo. Suppression of an autoinflammatory EAE (mouse MS model for CNS inflammation) has also been shown in vivo.

### Example 1

synNotch Induced Production of Suppressive Cytokine TGFb

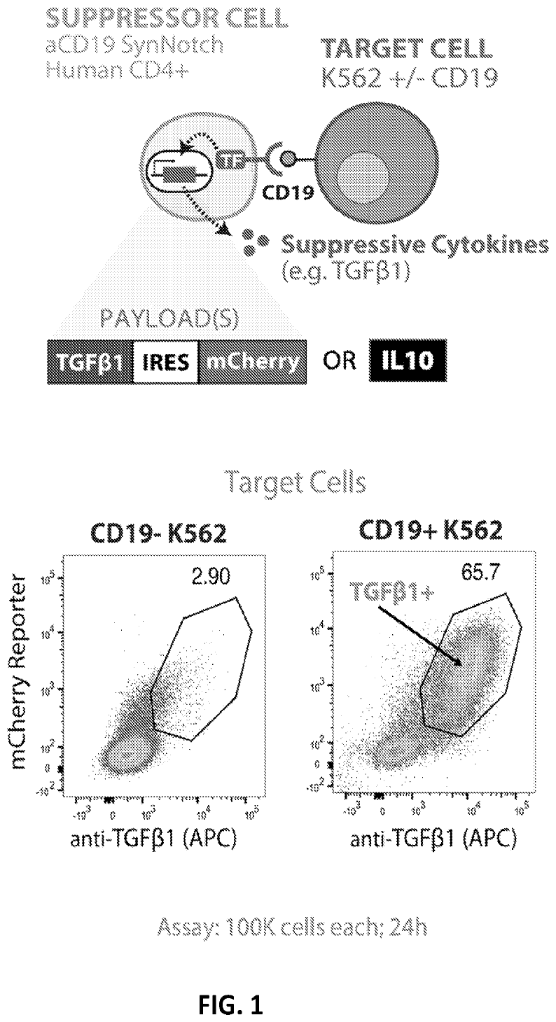

Engineered immune cells can produce immuno-suppressive payloads in response to a specific antigen. Human CD4⁺ T cells can selectively induce immune inhibitory cytokine TGFβ1 in response to CD19 antigen using SynNotch (measured by flow cytometry). The results shown in FIG. 1 show that expression of the suppressive cytokine TGFb can be induced by synNotch binding to CD19 on another cell.

### Example 2

Suppressor Cells that Produce Combination of TGFb (Suppressive Cytokine) and CD25 (IL2 Sink) are Very Effective at Suppressing CAR T Killing In Vitro.

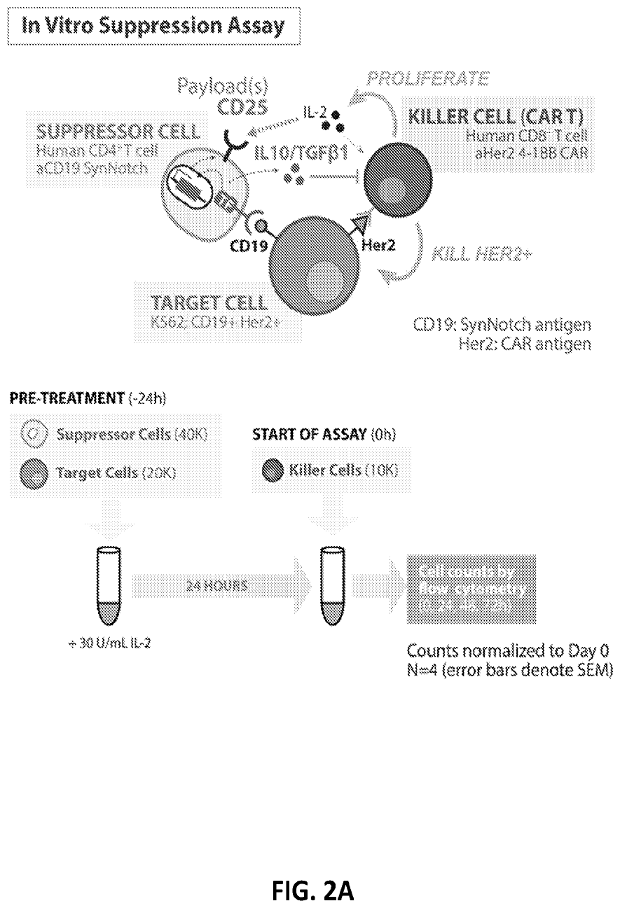

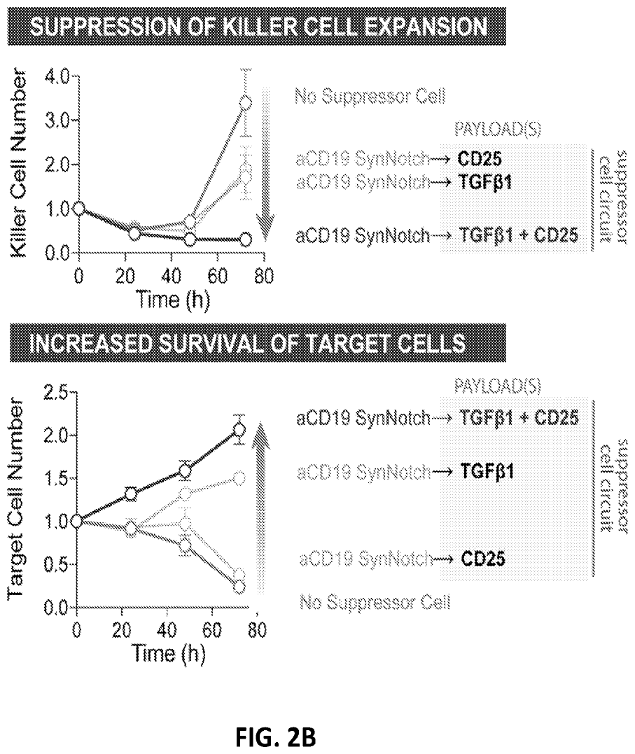

T cells inducibly producing a combination of inhibitory cytokine TGFβ1 and pro-inflammatory cytokine sink CD25 using synNotch show strong suppression of CAR T cell proliferation and killing in vitro. In vitro immune suppression was assayed by co-culturing three cells: (1) human CD4⁺ T cells with anti-CD19 SynNotch inducing production of CD25, TGFβ1, or both payloads, (2) K562 target cells expressing both a synNotch antigen, CD19, and a CAR antigen, Her2, and (3) human CD8+ T cells expressing an anti-Her2 4-1BB CAR. Cell counts were tracked over time using flow cytometry. Human CD4⁺ T cells with synNotch inducing production of both payloads is most effective at suppressing CAR T cell activity, proliferation of the CAR T cells and killing of the K562 target cells. These experiments and results are illustrated in FIGS. 2A and 2B.

### Example 3

Suppressor Cells that Produce Combination of IL10 (Suppressive Cytokine) and CD25 (IL2 Sink) are Very Effective at Suppressing CAR T Killing In Vitro.

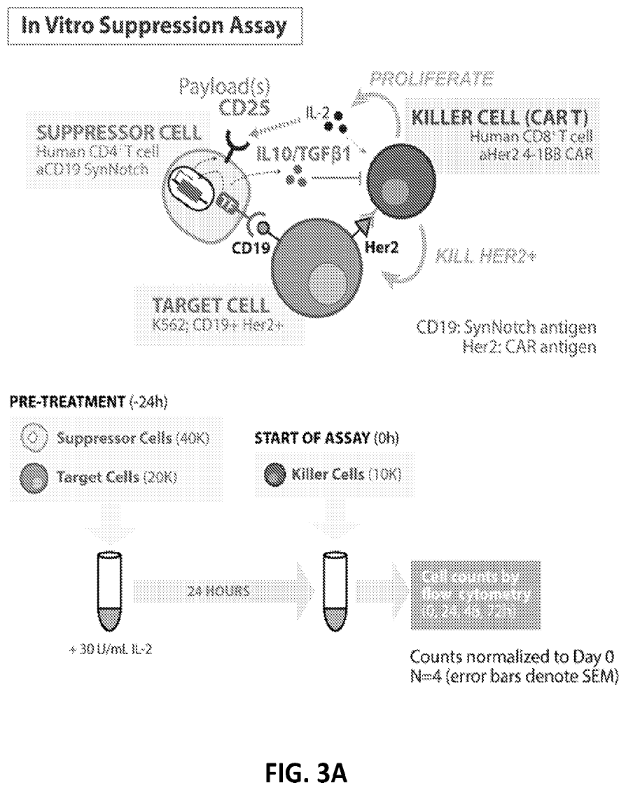

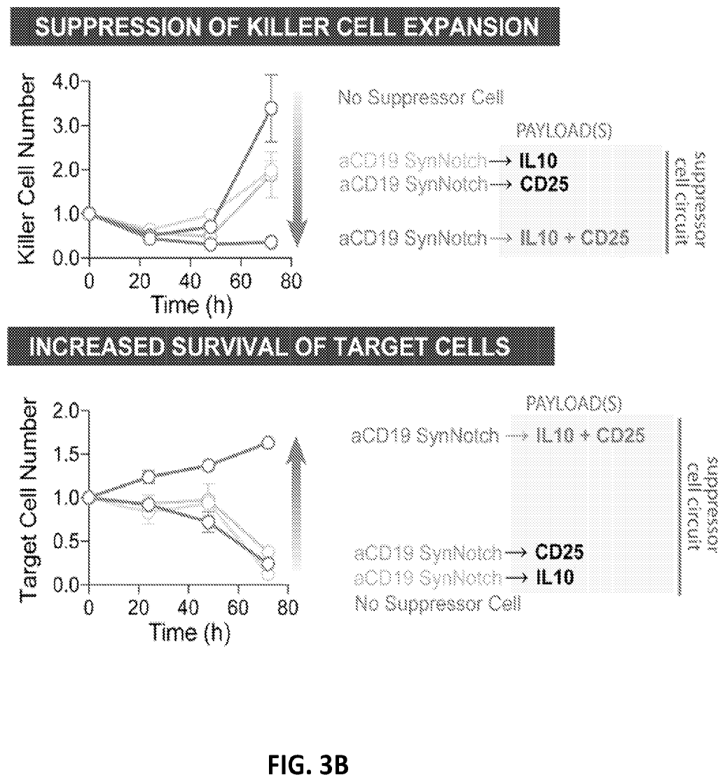

T cells inducibly producing a combination of inhibitory cytokine IL10 and pro-inflammatory cytokine sink CD25 using synNotch show strong suppression of CAR T cell proliferation and killing in vitro. In vitro immune suppression was assayed by co-culturing three cells: (1) human CD4+ T cells with anti-CD19 SynNotch inducing production of CD25, IL10, or both payloads, (2) K562 target cells expressing both a synNotch antigen, CD19, and a CAR antigen, Her2, and (3) human CD8+ T cells expressing an anti-Her2 4-1BB CAR. Cell counts were tracked over time using flow cytometry. Human CD4+ T cells with synNotch inducing production of both payloads is most effective at suppressing CAR T cell activity, proliferation of the CAR T cells and killing of the K562 target cells. These experiments and results are illustrated in FIGS. 3A and 3B.

### Example 4

Suppressor Cells that Produce Combination of TGFb (Suppressive Cytokine) and CD25 (IL2 Sink) are Very Effective at Suppressing CAR T Killing of Tumors In Vivo

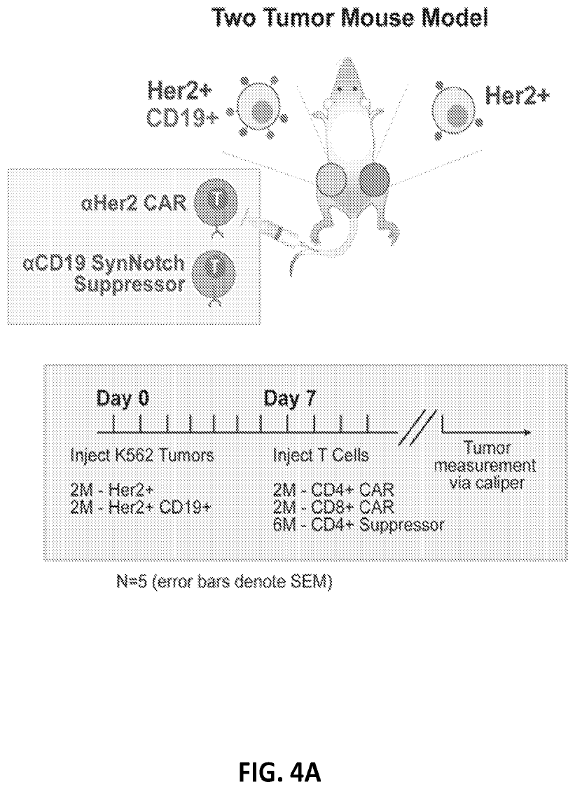

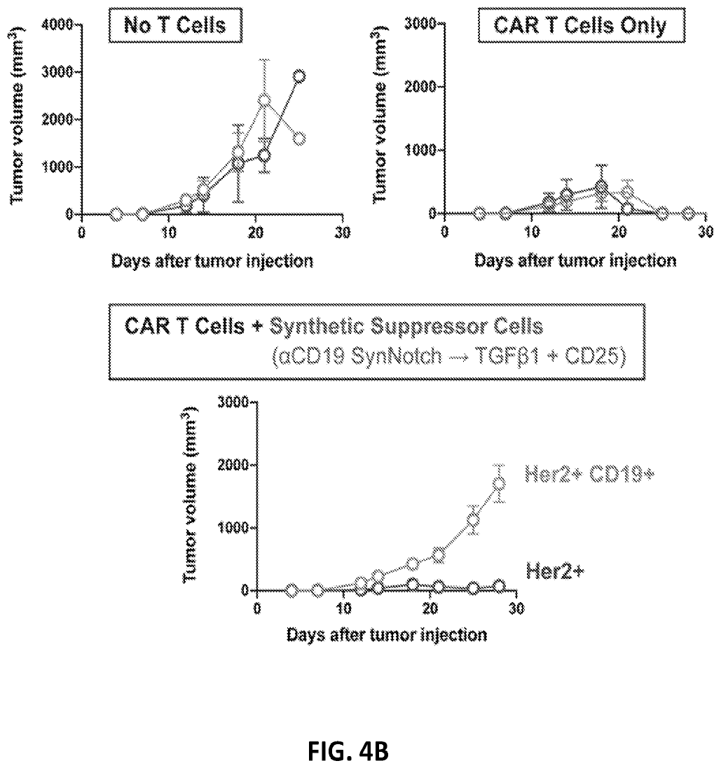

Synthetic immune-suppressive cells can locally suppress immune response. K562 tumors, Her2+ and Her2+CD19+, were subcutaneously injected in the flanks of N.S.G. mice. These mice were treated with either no T cells, anti-Her2 CAR T cells only, or anti-Her2 CAR T cells and synthetic suppressor cells (human CD4⁺ T cells with an anti-CD19 synNotch inducibly producing TGFβ1 and CD25) by i.v. injection after 7 days. Tumor volume was monitored by caliper measurement. Synthetic suppressor cells show strong local suppression of CAR T cell killing in the CD19+ tumor without suppressing CAR T cell killing of the CD19-tumor. These experiments and results are illustrated in FIGS. 4A and 4B.

### Example 5

### Engineered T Cells Overexpressing CD25 Increases Consumption of IL2 and Cell Proliferation.

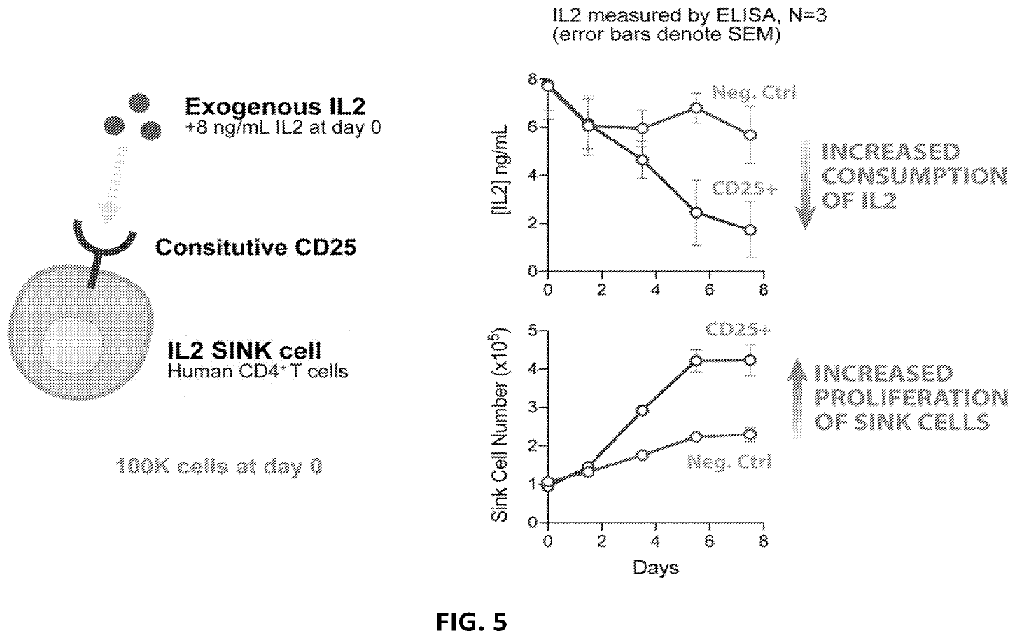

Human CD4⁺ T cells constitutively expressing CD25 show increased consumption of IL2 (measured by ELISA) and increased proliferation (measured by flow cytometry) in vitro when exogenous IL2 is added to the media. These experiments and results are illustrated in FIG. 5.

### Example 6

synNotch→IL10 Synthetic Suppressor Cells can Block Autoimmune Cell Proliferation in Brain and CNS in Mouse Neuroinflammation Model.

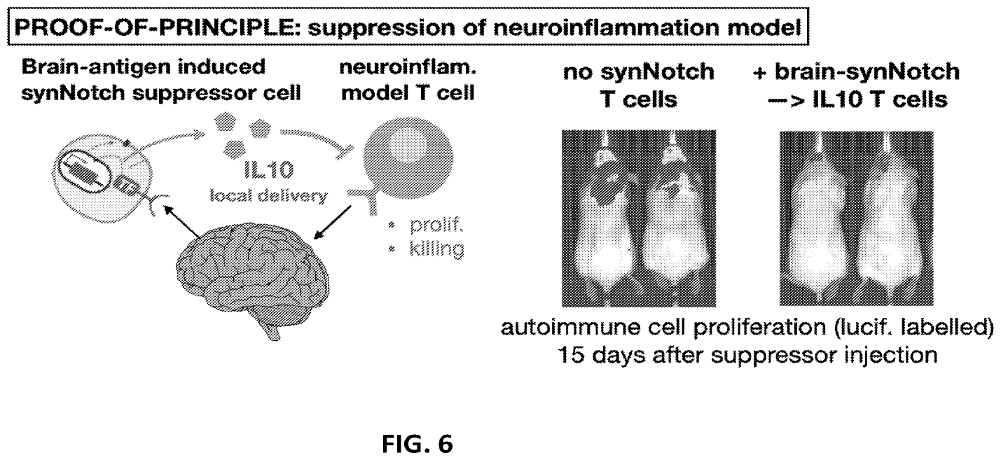

IL10 expression can be activated by mouse brain specific antigen (CDH10). These experiments and results are illustrated in FIG. 6.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An engineered immune cell comprising a molecular circuit comprising the following components:
(a) a binding-triggered transcriptional switch (BTTS); and
one or any combination of:
(b) a nucleic acid encoding a pro-inflammatory cytokine sink,
(c) a nucleic acid encoding an anti-inflammatory cytokine;
(d) a nucleic acid encoding an immune inhibitory receptor, or ligand thereof; and
(e) a nucleic acid encoding an ectonucleotidase;
wherein binding of the BTTS to a marker on the surface of a target cell activates expression of one or any combination of (b)-(e) in the cell.

2. The cell of claim 1, wherein the marker on the surface of a target cell is tissue and/or organ specific.

3. The cell of claim 2, wherein the marker on the surface of a target cell is CNS-specific.

4. The cell of any prior claim, wherein the anti-inflammatory cytokine is Il-1ra, IL-4, IL-6, IL-10, IL-11, IL-13, IL-35, and TGF-β, or a variant thereof.

5. The cell of any prior claim, wherein the cytokine sink comprises at least the extracellular domain of a receptor that binds to a pro-inflammatory cytokine.

6. The cell of claim 5, wherein the cytokine sink comprises at least the extracellular domain of IL-1R, IL-12R/CD25, IL-18R, TNFR1, TNFR2, IFNGR, GM-CSFR or a subunit thereof that binds to its cognate ligand.

7. The cell of any prior claim, wherein the cytokine sink is an antibody that is tethered to the cell and binds to a pro-inflammatory cytokine.

8. The cell of any prior claim, wherein the immune inhibitory receptor is PD1, CTLA4, BTLA, CD160, KRLG-1, 2B4, Lag-3, Tim-3, or TIGIT.

9. The cell of any prior claim, wherein the ectonucleotidase is CD39 or CD73.

10. The cell of any prior claim, wherein the circuit comprises components (a), (b) and (c).

11. The cell of any of claims 1-8, wherein the circuit comprises components (a) and (b).

12. The cell of any of claims 1-8, wherein the circuit comprises components (a) and (c).

13. The cell of any prior claim, wherein the BTTS comprises:
i. an extracellular binding domain that binds to a cell surface marker,
ii. a force sensing region,
iii. a transmembrane domain,
iv. one or more force-dependent cleavage sites that are cleaved when the force sensing region is activated, and
v. an intracellular domain comprising a transcriptional activator, where binding of the extracellular binding domain to the cell surface marker on the surface of another cell induces proteolytic cleavage of the one or more force-dependent cleavage sites to release the transcriptional activator, and
wherein the released transcriptional activator induces expression of the expression of one or any combination of (b)-(e) in the cell.

14. The cell of any prior claim, wherein the cell is a myeloid or lymphoid cell.

15. The cell of any prior claim, wherein the cell a CD4⁺ T cell or a macrophage.

16. A method of treating a subject, comprising:
administering to the subject a cell of any of claims 1-15.

17. The method of claim 16, wherein the subject has cancer, and the BTTS has an extracellular binding domain that binds to cells that are not part of the cancer.

18. The method of claim 16, wherein the subject is a recipient of an organ transplant, and the BTTS has an extracellular binding domain that binds to cells in the organ transplant.

19. The method of claim 16, wherein the subject has a cytokine-induced condition induced by an infectious disease, and the BTTS has an extracellular binding domain that binds to cells in the inflamed tissue.

20. The method of claim 16, wherein the subject has ARDS or organ-specific sepsis.

21. The method of claim 16, wherein the subject has an autoimmune disease.

22. The method of claim 16, wherein the subject has disease or condition that has local inflammation as part of its sequela.
